SEQUENCE LISTING (9 sequences)

SEQ ID NO: 1 - DNA, 24 bp, synthetic construct
  misc_feature 1..24: note = Synthetic Oligonucleotide 2006-PTO
  source 1..24: mol_type = other DNA
tcgtcgttttgtcgttttgtcgtt

SEQ ID NO: 2 - DNA, 37 bp, synthetic construct
  misc_feature 1..37: note = Synthetic Oligonucleotide 2006-3dT4G5T4
  source 1..37: mol_type = other DNA
tcgtcgttttgtcgttttgtcgttttttgggggtttt

SEQ ID NO: 3 - DNA, 28 bp, synthetic construct
  misc_feature 1..28: note = Synthetic Oligonucleotide 2006-T4-PDE
  source 1..28: mol_type = other DNA
tcgtcgttttgtcgttttgtcgtttttt

SEQ ID NO: 4 - DNA, 26 bp, synthetic construct
  misc_feature 1..26: note = Synthetic Oligonucleotide 2007-PDE-T4
  source 1..26: mol_type = other DNA
tcgtcgttgtcgttttgtcgtttttt

SEQ ID NO: 5 - DNA, 35 bp, synthetic construct
  misc_feature 1..35: note = Synthetic Oligonucleotide 2007-T4G5T4
  source 1..35: mol_type = other DNA
tcgtcgttgtcgttttgtcgttttttgggggtttt

SEQ ID NO: 6 - DNA, 28 bp, synthetic construct
  misc_feature 1..28: note = Synthetic Oligonucleotide TCG8-T4
  source 1..28: mol_type = other DNA
tcgtcgtcgtcgtcgtcgtcgtcgtttt

SEQ ID NO: 7 - DNA, 29 bp, synthetic construct
  misc_feature 1..29: note = Synthetic Oligonucleotide 2006-G5
  source 1..29: mol_type = other DNA
tcgtcgttttgtcgttttgtcgttggggg

SEQ ID NO: 8 - DNA, 37 bp, synthetic construct
  misc_feature 1..37: note = Synthetic Oligonucleotide TCG8-T4G5T4
  source 1..37: mol_type = other DNA
tcgtcgtcgtcgtcgtcgtcgtcgttttgggggtttt

SEQ ID NO: 9 - DNA, 13 bp, synthetic construct
  misc_feature 1..13: note = Synthetic Oligonucleotide PDE-2006
  source 1..13: mol_type = other DNA
ttttgggggtttt

(19) United States
(12) Patent Application Publication
(10) Pub. No.: US 2026/0327630 A1
(43) Pub. Date: Oct. 22, 2026

(54) IMMUNOSTIMULATORY OLIGONUCLEOTIDES

(71) Applicant: Elanco Animal Health GmbH, Monheim am Rhein (DE)

(72) Inventor: Thomas ILG, Meerbusch (DE)

(21) Appl. No.: 19/727,994

(22) Filed: Jul. 1, 2026

Related U.S. Application Data
(63) Continuation of application No. 18/366,306, filed on Aug. 7, 2023.
Application No. 18/366,306 is a division of application No. 16/772,541, filed on Jun. 12, 2020, now Pat. No. 11,932,857.

(30) Foreign Application Priority Data
Dec. 15, 2017 (EP) 17207740.6
Dec. 15, 2017 (EP) 17207746.3
Dec. 15, 2017 (EP) 17207750.5

(51) Int. Cl. C12N 15/117; A61K 39/00; A61K 39/39; A61P 37/04

(52) U.S. Cl. CPC C12N 15/117; A61K 39/39; A61P 37/04; A61K 2039/55555; A61K 2039/55561; A61K 2039/60; C12N 2310/17; C12N 2310/315; C12N 2310/3341; C12N 2310/3515; C12N 2320/31

(57) ABSTRACT

Compositions and methods for stimulating toll-like receptor 9 (TLR9) are provided. More particularly, immunostimulatory oligonucleotides, methods of enhancing immunostimulatory properties of oligonucleotides, and methods of eliciting immune responses are disclosed herein.

### REFERENCE TO RELATED APPLICATIONS

This patent application is a continuation patent application of U.S. patent application Ser. No. 18/366,306, filed 7 Aug. 2023, which is a divisional patent application of U.S. patent application Ser. No. 16/772,541, filed 12 Jun. 2020, now U.S. Pat. No. 11,932,857, issued 19 Mar. 2024, which is a national stage patent application of International Patent Application No. PCT/EP2018/084019, filed 7 Dec. 2018, which claims priority to European Patent Application Nos. EP17207740.6, EP17207746.3, and EP17207750.5, all filed on 15 Dec. 2017, the disclosures of each of which are incorporated herein by reference in their entireties.

### SEQUENCE LISTING

Pursuant to the PatentCenter legal framework and 37 C.F.R. §§ 1.821-1.825 (See MPEP 2442.03(a)), a Sequence Listing in the form of an ASCII-complaint xml file (entitled “Sequence_Listing_2920951-177002_ST26.xml” created on 21 May 2026, and 12,674 bytes in size) is submitted concurrently with the instant patent application and the entire contents of the Sequence Listing are herein incorporated by reference.

### BACKGROUND

### Field

Compositions and methods for stimulating toll-like receptor 9 (TLR9) are provided. More particularly, immunostimulatory oligonucleotides, methods of enhancing immunostimulatory properties of oligonucleotides, and methods of eliciting immune responses are disclosed herein.

### Description of Related Art

Antibiotic resistance is a global problem negatively affecting numerous industries. Methicillin resistant Staphylococcus aureus (MRSA) and other “super bugs” are creating havoc in hospitals and doctors' offices, making visits to health centers potentially lethal. The agriculture industry sees similar issues. Entire herds are at risk of pathogenic infection due to limited space and non-sterile environments. One sick cow, for example, in close proximity to her herd can exponentially increase morbidity and mortality rates. Despite the risk of infections, antibiotic treatments are becoming more disfavored due to increased costs and consumers demanding meats and dairy products that have not been exposed to antibiotics. And those producers who do use antibiotic therapies understand that even broad spectrum antibiotics are not entirely effective against every pathogen that may come in contact with a herd.

### SUMMARY

Thus, there is a need for non-antibiotic based therapies for treating or preventing infection in animals. The disclosed compositions and methods are directed to these and other important needs.

Disclosed herein are immunostimulatory oligonucleotides comprising at least one CpG motif and a 3′ cholesteryl moiety.

Immunostimulatory compositions comprising immunostimulatory oligonucleotides are also provided herein.

Also disclosed are methods for enhancing the immunogenicity of a TLR9 ligand comprising attaching a cholesteryl moiety to the 3′ terminus of the TLR9 ligand via a linker, wherein the TLR9 ligand is an oligonucleotide having at least one CpG motif.

Methods are also provided for eliciting a TLR9-mediated immune response in a subject comprising administering to the subject any one of the immunostimulatory oligonucleotides or the immunostimulatory compositions described herein.

### BRIEF DESCRIPTION OF THE DRAWINGS

The summary, as well as the following detailed description, is further understood when read in conjunction with the appended drawings. For the purpose of illustrating the disclosed compositions and methods, there are shown in the drawings exemplary embodiments of the compositions and methods; however, the compositions and methods are not limited to the specific embodiments disclosed. In the drawings:

FIG. 1 depicts the chemical structure of a cholesteryl moiety attached to a hexanediol linker.

FIGS. 2A and 2B compare the TLR9 stimulatory activity of oligonucleotide PTO-2006, oligonucleotide PDE-2006 having a 3′ TTTTGGGGGTTTT (SEQ ID NO:9) sequence (“2006-3dT4G5T4”), and oligonucleotide 2006-3dT4G5T4 having a 3′ cholesteryl moiety attached via a hexanediol linker shown in FIG. 1 (“2006-3dT4G5T43C”) in HEKBlue-hTLR9 cells.

FIG. 3 depicts the ability to elicit a TLR9-mediated immune response in HEKBlue-hTLR9 cells of oligonucleotide PDE-2006 having a 3′ TTTT sequence (“2006-T4-PDE”) and oligonucleotide 2006-T4-PDE with a cholesteryl moiety attached via a hexanediol linker of FIG. 1 to the oligonucleotide's 3′ terminus (“3Chol-2006-T4-PDE”).

FIGS. 4A and 4B compare the TLR9 stimulatory characteristics of oligonucleotide 2006-3dT4G5T4 and oligonucleotide 2006-3dT4G5T4C.

FIG. 5 compares the TLR9 stimulatory characteristics of oligonucleotide 2006-3dT4G5T4 and oligonucleotide 2006-3dT4G5T4C.

FIG. 6 depicts the chemical structure of a cholesteryl moiety attached to a hexaethylene glycol linker.

FIG. 7 compares the TLR9 stimulatory ability of oligonucleotide 2006-T4G5T4 and oligonucleotide 2006-T4G5T4 with the cholesteryl-linker moiety of FIG. 6 attached to its 3′ terminus (“2006-T4G5T4-3Chol” or “2006-T4G5T4-3C”).

FIGS. 8A and 8B compare the immunogenicity of oligonucleotides with and without 3′ cholesteryl modification. FIG. 8A compares the ability of oligonucleotide 2007-PDE-T4oligonucleotide to elicit a TLR9-mediated immune response in HEKBlue-hTLR9 cells to that of oligonucleotide 2007-PDE-T4 oligonucleotide with a cholesteryl moiety attached via a hexanediol linker of FIG. 1 (“2007-PDE-T4-3Ch”), and FIG. 8B compares the ability of oligonucleotide 2007-PDE-T4 having a 3′ GGGGGTTTT sequence (“2007-T4G5T4”), and oligonucleotide 2007-T4G5T4 with a cholesteryl moiety attached via a hexanediol linker shown in FIG. 1 (“2007-T4G5T4-3Ch”) in HEKBlue-hTLR9 cells.

FIGS. 9A and 9B compare the immunogenicity of oligonucleotides with and without 3′ cholesteryl modification. More specifically, FIG. 9A illustrates the immunogenicity of 2006-PTO, 2006-3dT4G5T4, and 2006-3dT4G5T4C oligonucleotides via a TLR9-mediated immune response in Ramos-Blue cells, and FIG. 9B illustrates the results depicted in FIG. 9A over narrower concentration range.

FIGS. 10A and 10B compare the ability of oligonucleotides with or without a 3′ cholesteryl moiety attached via a hexanediol linker to elicit a TLR9-mediated immune response in Ramos-Blue cells. More specifically, FIG. 10A illustrates the relative ability of oligonucleotides 2006-3dT4G5T4 and 2006-3dT4G5T4C to elicit a TLR9-mediated immune response. FIG. 10B illustrates the results of FIG. 10A over a narrower concentration range.

FIG. 11 compares the immunogenicity of oligonucleotide 2006-T4-PDE and oligonucleotide 2006-T4-PDE having a 3′ cholesteryl moiety attached via a hexanediol linker shown in FIG. 1 (“3Chol-2006-T4-PD”) in Ramos-Blue.

FIG. 12 compares ability to elicit a TLR9-mediated immune response in Ramos-Blue cells of oligonucleotide 2006-3dT4G5T4 and oligonucleotide 2006-3dT4G5T4-3Chol.

FIGS. 13A, 13B, and 13C compare the abilities of 2007-PDE-T4, 2007-T4G5T4, and TCG8-T4 oligonucleotides, with or without a cholesteryl moiety attached to the 3′ terminus of the oligonucleotide, to stimulate TLR9 in Ramos-Blue cells. FIG. 13A illustrates the different immunogenicities of oligonucleotides 2007-PDE-T4 and 2007-PDE-T4-Ch3. FIG. 13B illustrates the different immunogenicities of oligonucleotides 2007-T4G5T4 and 2007-T4G5T4-3Ch. FIG. 13C illustrates the different immunogenicities of oligonucleotides TCG8-T4 and TCG8-T4-Ch3.

FIGS. 14A, 14B, 14C, and 14D compare stimulatory activity of several oligonucleotides and cholesteryl-modified oligonucleotides on mouse TLR9 (“mTLR9”) in HEKBlue-mTLR9 cells.

FIG. 14A compares the ability to elicit a TLR9-mediated response in HEKBlue-mTLR9 cells of an unmodified 2007-PDE-T4 oligonucleotide and oligonucleotide 2007-PDE-T4-3Ch. FIG. 14B compares the ability of an unmodified 2007-T4G5T4 oligonucleotide and oligonucleotide 2007-T4G5T4-3Ch to elicit a TLR9-mediated response in HEKBlue-mTLR9 cells. FIG. 14C compares the ability of oligonucleotide TCG8-T4 and oligonucleotide TCG8-T4-3Ch to elicit a TLR9-mediated response in HEKBlue-mTLR9 cells; and FIG. 14D compares the ability of TCG8-T4G5T4 oligonucleotide and oligonucleotide TCG8-T4G5T4 having a 3′ cholesteryl moiety attached via a hexanediol linker shown in FIG. 1 (“TCG8-T4G5T4-3Ch”) to elicit a TLR9-mediated response in HEKBlue-mTLR9 cells.

FIG. 15 depicts the chemical structure of a cholesteryl moiety attached to a hexaethylene glycol linker.

FIGS. 16A, 16B, 16C, and 16D depict the effect of modifying either the 3′ or 5′ termini of oligonucleotides with a cholesteryl moiety on the oligonucleotide's ability to elicit a TLR9-mediated immune response. FIG. 16A graphically depicts the ability to stimulate TLR9 in HEKBlue-hTLR9 cells of oligonucleotides 2006-PDE-T4, 2006-PDE-T4-Chol, and oligonucleotide 2006-PDE-T4 having a 5′ cholesteryl moiety attached via a hexaethylene glycol linker shown in FIG. 15 (“2006-PDE-T4-5Chol”). FIG. 16B graphically depicts the ability to stimulate TLR9 in HEKBlue-hTLR9 cells of oligonucleotide 2006-PTO, oligonucleotide 2006-PDE having a GGGGG 3′ terminal sequence (“2006-G5”), oligonucleotide 2006-G5 having a 3′ cholesteryl moiety attached via a hexaethylene glycol linker shown in FIG. 6 (“2006-G5-3Chol”), and oligonucleotide 2006-G5 having a 5′ cholesteryl moiety attached via a hexaethylene glycol linker shown in FIG. 15 (“2006-G5-5Chol”). FIG. 16C graphically depicts the ability to stimulate TLR9-mediated immune responses in HEKBlue-hTLR9 cells of oligonucleotides 2006-PTO, 2006-T4G5T4, 2006-T4G5T4-3Chol, and oligonucleotide 2006-T4G5T4 having a 5′ cholesteryl moiety attached via a hexaethylene glycol linker shown in FIG. 15 (“2006-T4G5T4-5Chol”). FIG. 16D graphically depicts the ability to stimulate TLR9-mediated immune responses in HEKBlue-hTLR9 cells of oligonucleotide TCG8-T4G5T4, oligonucleotide TCG8-T4G5T4 having a 3′ cholesteryl moiety attached via a hexaethylene glycol linker shown in FIG. 6 (“TCG8-T4G5T4-3Chol”), and oligonucleotide TCG8-T4G5T4 having a 5′ cholesteryl moiety attached via a hexaethylene glycol linker shown in FIG. 15 (“TCG8-T4G5T4-5Chol”).

FIGS. 17A, 17B, 17C, and 17D depict the effect of modifying the 3′ or 5′ termini of oligonucleotides with a cholesteryl moiety. FIG. 17A graphically depicts the ability to stimulate TLR9 in Ramos-Blue cells of oligonucleotides 2006-PDE-T4, 2006-PDE-T4-Chol, and 2006-PDE-T4-5Chol. FIG. 17B graphically depicts the ability to stimulate TLR9 in Ramos-Blue cells of oligonucleotide 2006-PTO, 2006-G5, 2006-G5-3Chol, and 2006-G5-5Chol. FIG. 17C graphically depicts the ability to stimulate TLR9-mediated immune responses in Ramos-Blue cells of oligonucleotides 2006-PTO, 2006-T4G5T4, 2006-T4G5T4-3Chol, and oligonucleotide 2006-T4G5T4-5Chol. FIG. 17D graphically depicts the ability to stimulate TLR9-mediated immune responses in Ramos-Blue cells of oligonucleotides TCG8-T4G5T4, TCG8-T4G5T4-3Chol, and TCG8-T4G5T4-5Chol.

### DETAILED DESCRIPTION OF A PREFERRED EMBODIMENT

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

When a range of values is expressed, another embodiment includes from the one particular value and/or to the other particular value. Further, reference to values stated in ranges includes each and every value within that range. All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

As used herein, “CpG motif” refers to a cytosine-guanine dinucleotide sequence. The immunostimulatory nucleic acids described herein contain one or more CpG motifs, which when unmethylated can interact with toll-like receptor proteins (TLRs) and elicit an immune response.

The term “subject” as used herein is intended to mean any animal, in particular, mammals, and any type of avian, mammalian, or aquatic species can be treated using the disclosed methods.

Various terms relating to aspects of the description are used throughout the specification and claims. Such terms are to be given their ordinary meaning in the art unless otherwise indicated. Other specifically defined terms are to be construed in a manner consistent with the definitions provided herein.

Disclosed herein are immunostimulatory oligonucleotides comprising at least one CpG motif and a 3′-terminal cholesteryl moiety. It has been previously shown that CpG motifs in oligodeoxynucleotides (ODN) can elicit an immune response in mammals. In some instances, the CpG motif is recognized by a toll-like receptor (TLR). Examples of CpG-recognizing TLRs include, but are not limited to, mammalian homologs of TLR9. Thus in some aspects of the present disclosures, the CpG-recognizing TLR is a mouse, human, cow, pig, horse, or sheep TLR9 homolog. The immunogenicity of an ODN may not be sufficient to elicit immune responses capable of warding off infection in susceptible populations or infected individuals. As demonstrated herein, the immunostimulatory properties of an ODN can be enhanced by modifying oligonucleotides, especially with the addition of a thymine run, a guanine run, and/or a cholesteryl moiety at the 3′ terminus of the ODN.

The immunostimulatory oligonucleotides of the present disclosure comprise at least one CpG motif. In some embodiments, the immunostimulatory oligonucleotides comprise between one and ten CpG motifs. In other embodiments the immunostimulatory oligonucleotides can comprise even twenty CpG motifs. Thus, in some embodiments, the immunostimulatory oligonucleotides of the present disclosure comprise one, two, three, four, five, six, seven, eight, nine, or ten CpG motifs. In other embodiments, the immunostimulatory oligonucleotides comprise between eleven and fifteen CpG motifs or even between fifteen and twenty CpG motifs.

Oligonucleotides comprising phosphodiester and/or phosphorothioate linkages between nucleotides are contemplated herein. In some aspects, the oligonucleotides of the present disclosure comprise phosphodiester linkages between the oligonucleotide's nucleotides. In other aspects, the oligonucleotides comprise phosphorothioate linkages between the oligonucleotide's nucleotides. Other linkages are also contemplated herein. For example, the oligonucleotide of the present disclosure may comprise other linkages including, but not limited to, phosphoacetate, methylphosphonate, and phosphonocarboxylate linkages. Some of the linkages may provide desirable advantages over the other linkages, such as cost of production, ease and/or quality of production, and enhanced immunostimulatory impact.

In some aspects of the present disclosure, the oligonucleotide's immunogenicity due to the CpG motifs may be further enhanced by non-CpG sequences. As shown in the Examples, the addition of a thymine run to the 3′ terminus of the oligonucleotide can improve the ability of the oligonucleotide to elicit a TLR9-mediated immune response. For this reason, in some embodiments of the present disclosures the 3′ terminal sequence of the immunostimulatory oligonucleotide comprises a plurality of thymine nucleotides as the 3′ terminal sequence. In some aspects, this plurality of thymine nucleotide comprises consecutive thymine nucleotides. In some aspects, the plurality of thymine nucleotides comprises between four and six consecutive thymine nucleotides. For example, in some embodiments of the present disclosure, the 3′ terminal sequence comprises SEQ ID NO:9. In some embodiments, the oligonucleotide comprises SEQ ID NO: 2, 3, 4, 5, 6, or 8. And in some aspects, the 3′ terminal sequence of the oligonucleotide sequence is TTTT.

Other sequence modifications to the 3′ end of the immunostimulatory oligonucleotide may also contribute to enhanced immunogenicity. For example, in some embodiments of the present disclosures, the immunostimulatory oligonucleotide comprises a plurality of guanine nucleotides at or near the 3′ terminal sequence. In some aspects, the 3′ terminal sequence of the immunostimulatory oligonucleotide comprises a plurality of guanine nucleotides. In some aspects, the plurality of guanine nucleotides comprises consecutive guanine nucleotides, such as an oligonucleotide according to the present disclosure having a 3′ terminal sequence of GGGGG. In some aspects, the oligonucleotide comprises SEQ ID NO:7.

Increasing immunogenicity of an immunostimulatory oligonucleotide is not limited to modifications of the 3′ terminus of the oligonucleotide. Internal sequences can also be modified, for example, to increase the number of CpG motifs. In some aspects, oligonucleotides can be synthesized comprising additional CpG motifs between the 5′ and 3′ ends of the oligonucleotide. In some aspects, the immunostimulatory oligonucleotide comprises the sequence (TCG)ₙ, where n is between three and ten. Thus, in some aspects of the present disclosure, the oligonucleotide comprises the sequence (TCG)ₙ, where n is 3, 4, 5, 6, 7, 8, 9, or 10.

In some embodiments of the present disclosure, the immunostimulatory oligonucleotide may comprise a lipid moiety at the 3′ terminus to enhance the immunogenic properties of the oligonucleotide. Thus, in some embodiments, a cholesteryl moiety is covalently attached to the 3′-terminal nucleotide of the immunostimulatory oligonucleotide via a linker. The cholesteryl moiety likely increases the oligonucleotide's immunogenicity by preventing degradation, increasing solubility, generating ligand multivalency by forming higher order structures (e.g., micelles), increasing the stability of the oligonucleotide in a pharmaceutical composition, or any combination thereof. The linker, having at least two moieties capable of forming covalent bonds, can bond with the cholesteryl moiety and with the oligonucleotide. For example, in some embodiments the linker interacts with the cholesteryl moiety's hydroxyl group to form a covalent bond and with the 3′ terminal nucleotide of an oligonucleotide. In some aspects, the cholesteryl moiety is covalently bound to the linker to form a cholesteryl-linker moiety. In some aspects, the linker is first attached to the cholesteryl moiety and the resulting cholesteryl-linker is then attached to the oligonucleotide. In other aspects, the linker is first attached to the oligonucleotide and then to the cholesteryl moiety. In some aspects, the cholesteryl-linker is commercially available.

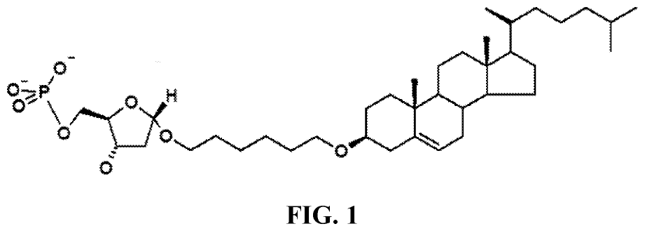

In addition to having moieties that can bind to the oligonucleotide and the cholesteryl moiety, some embodiments of the linker comprise a carbon chain, and in some aspects the carbon chain comprises between 3 and 12 carbon atoms. Diols, for example, can be used as a linker between the cholesteryl moiety and the oligonucleotide as the terminal hydroxyl groups can covalently bond with the oligonucleotide's and the cholesteryl moiety's hydroxyl groups. In some aspects, the linker comprises a hexanediol. In some aspects, a cholesteryl-linker moiety has the chemical structure depicted in FIG. 1. Other embodiments provide for a linker comprising a repeated chemical unit. The chemical unit, in some aspects, is repeated between two and twelve times. In some aspects the repeated chemical unit comprises ethylene glycol, and when the ethylene glycol chemical unit is repeated six times, the linker comprises a hexaethylene glycol. A linker comprising hexaethylene glycol may have the chemical structure depicted in FIG. 6.

In some circumstances it will be desirable to deliver an oligonucleotide as described herein to a subject in need thereof. The oligonucleotide may be delivered as an immunostimulatory composition. Immunostimulatory compositions comprising any of the oligonucleotides disclosed herein are provided. These immunostimulatory compositions, in some aspects, comprise the oligonucleotide as well as other components that affect the immunogenicity, effectiveness, and efficiency of the composition. In some embodiments of the present disclosure the immunostimulatory composition may include in addition to the immunostimulatory oligonucleotide a vaccine for preventing or treating an infectious disease, a vector for delivering the oligonucleotide to the subject, a pharmaceutical carrier, or any combination thereof. For example, in some aspects the oligonucleotide is packaged in a viral vector that allows for the targeted delivery of the oligonucleotide. The oligonucleotide may, in some aspects, be added to a cationic liposomal delivery vehicle to enhance the ability of the oligonucleotide to traverse lipid cell membranes and/or membranes of cellular organelles containing TLR9.

Infectious diseases that may be treated or prevented by administration of the immunostimulatory oligonucleotides or immunostimulatory compositions described herein include, but are not limited to, viral, bacterial, fungal, helminthic, or other parasitic infection. It is contemplated that administering the immunostimulatory oligonucleotides or compositions of the present disclosure results in an immune response that creates an environment hostile to an invading pathogen. Therefore, invading pathogens may be unable to establish an infection sufficient to result in a negatively altered health state in the host organism. The administration of the immunostimulatory oligonucleotides and/or compositions may provide a non-antigen-specific immune response that augments or works in parallel with an antigen-specific immune response against an invading pathogen.

In some aspects, the immunostimulatory composition comprising the oligonucleotide may further comprise a vaccine for preventing or treating an infectious disease. The combination of the oligonucleotide and the vaccine may be done for efficiency reasons as delivering multiple pharmaceuticals separately adds to the cost of treatment. The oligonucleotide and the vaccine may also be delivered as a single immunostimulatory composition to elicit a non-antigen specific immune response against any current infections as well as to initiate the development of an antigen specific immune response.

Also contemplated herein are immunostimulatory compositions that comprise an oligonucleotide as described herein and a pharmaceutically acceptable carrier. In some aspects, the pharmaceutically acceptable carrier is any pharmaceutically acceptable carrier. The pharmaceutically acceptable carrier adapts the composition for administration by a route selected from intravenous, intramuscular, intramammary, intradermal, intraperitoneal, subcutaneous, by spray, by aerosol, in ovo, mucosal, transdermal, by immersion, oral, intraocular, intratracheal, intranasal, pulmonary, rectal, or other means known to those skilled in the art. The pharmaceutically acceptable carrier(s) may be a diluent, adjuvant, excipient, or vehicle with which the immunostimulatory composition is administered. Such vehicles may be liquids, such as water and oils, including those of petroleum, animal, vegetable, or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the molecules of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g., Remington: The Science and Practice of Pharmacy, 21^{st} Edition, Troy, D. B. ed., Lippincott Williams and Wilkins, Philadelphia, PA 2006, Part 5, Pharmaceutical Manufacturing pp 691-1092 (see especially pp. 958-989).

In some embodiments, the oligonucleotide and the carrier are coupled, e.g., chemically coupled. As used to describe the relationship between the oligonucleotide and the carrier, “coupled” refers to physical association of the oligonucleotide and the carrier. When the oligonucleotide and the carrier are bound to each other, interact with each other, or are combined, or otherwise joined, they can be deemed to be coupled.

The immunostimulatory compositions described herein further comprise a hapten in some embodiments. In some aspects, the immunostimulatory oligonucleotide is linked to the hapten. The hapten may elicit an immunoresponse against a specific microorganism, such as E. coli or Salmonella, while the immunostimulatory oligonucleotide elicits a non-specific immunoresponse mediated by TLR9 interaction with the oligonucleotide. These and other infectious microorganisms are of particular interest to large agricultural producers, such as cattle, sheep, and pig producers.

Methods are also provided for enhancing the immunogenicity of a TLR9 ligand comprising attaching a cholesteryl moiety to the ligand, wherein the ligand is an immunostimulatory oligonucleotide having at least one CpG motif and wherein the cholesteryl moiety is attached, via a linker, to the 3′ terminal nucleotide of the oligonucleotide.

Other methods disclosed herein provide for eliciting a TLR9-mediated immune response in a subject in need thereof comprising administering to the subject an oligonucleotide having a plurality of CpG motifs and a cholesteryl-linker moiety attached to the 3′ terminal nucleotide of the oligonucleotide. In some aspects of the methods for eliciting a TLR9-mediated immune response, the oligonucleotide is administered as an immunostimulatory composition.

The subject to which the immunostimulatory oligonucleotide or immunostimulatory composition is administered, in some embodiments of this disclosure, is an animal. In some aspects, the animal is at a heightened risk of infection by a pathogen and especially a pathogen having a CpG-based pathogen associated molecular pattern (PAMP). When an immunostimulatory oligonucleotide and/or immunostimulatory composition is administered to such an animal, a TLR9-mediated immune response will assist in preventing infection by the pathogen or alleviation of symptoms caused by the pathogen. It will be understood by those skilled in the art that the immunostimulatory oligonucleotides of the present invention need not be specific for a particular pathogen, but rather, stimulate a non-antigen specific immune response. The oligonucleotides also need not be specific for a particular animal. Thus, in some aspects of the present disclosure, the subject is a mammal. In some aspects the subject is a herd or farm animal such as a pig, cow, horse or sheep. Administration to herd animals may help prevent the spread of infection to large populations of animals in crowded conditions such as pens and/or sharing common feed or water sources. The oligonucleotides of the present disclosure provide a distinct advantage over traditional forms of prophylactic treatment of infection in that the use of antibiotics is growing more disfavored, especially with the emergence of bacterial resistance to antibiotic therapy.

In some embodiments, the subject may be a human. As with herd animals, resistance to antibiotics consumed by humans is becoming common in bacteria, and treatment options for resistant infections are limited. The oligonucleotides and methods of the present disclosure provide a much needed solution to so-called “super-bugs” such as methicillin-resistant Staphylococcus aureus.

It is also contemplated herein that the subject to which the immunostimulatory oligonucleotide or composition is administered to may be a mouse, rat, hamster, gerbil, or other rodent. The subject may also be nonmammalian. For example, the subject, in some aspects, is an aquatic species.

### EXAMPLES

The following examples are provided to further describe some of the embodiments disclosed herein. The examples are intended to illustrate, not to limit, the disclosed embodiments.

### Example 1: 3′-Cholesteryl Modification of ODNs Results in Strongly Increased TLR9 Stimulatory Activity

### Human TLR9, Recombinant Overexpression in HEKBlue

### 3′-Cholesteryl Modification of PDE-ODNs (I)

3′-cholesteryl modification (see FIG. 1 for chemical structure of the cholesteryl-linker moiety) was applied to a PDE-ODN (Table 1, 2006-3dT4G5T4) that has fair activity on human TLR9. The modified and unmodified forms were tested in vitro in HEKBlue-hTLR9 cells (Invivogen), a cell line expressing a human TLR9.

TABLE 1
ODN sequences (lower case: PTO bonds)

| ODN | SEQ ID NO | Sequence |
|---|---|---|
| 2006-PTO | SEQ ID | tcgtcgttttgtcg |
| | NO: 1 | ttttgtcgtt |
| 2006- | SEQ ID | TCGTCGTTTTGTCGTTTT |
| 3dT4G5T4 | NO: 2 | GTCGTTTTTTGGGGGTTTT |
| 2006- | SEQ ID | TCGTCGTTTTGTCGTTTTG |
| 3dT4G5T43C | NO: 2 | TCGTTTTTTGGGGGTTTTX |

X = 3′-Cholesteryl

TABLE 2
Half maximal effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-PTO | 26.0 | 56 |
| 2006-3dT4G5T4 | 404 | 120 |
| 2006-3dT4G5T43C | 13.6 | 103 |

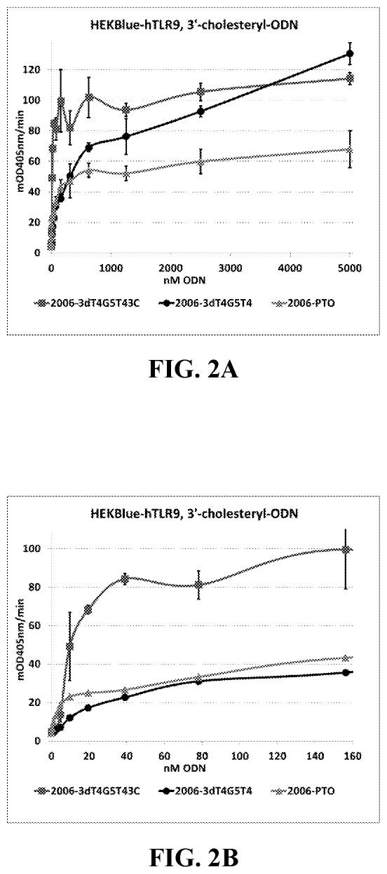

The results suggest that the TLR9-stimulatory activity of 2006-3dT4G5T4 improves considerably upon 3′-cholesteryl modification with respect to the EC₅₀, which is almost 30-fold lower for 2006-3dT4G5T43C (Table 2, FIGS. 2A, 2B).

### 3′-Cholesteryl Modification of PDE-ODNs (II)

A cholesteryl moiety (see FIG. 1 for the chemical structure of the cholesteryl-linker moiety) was attached to the 3′ terminal nucleotide of 2006-T4-PDE (SEQ ID NO:3, Table 3) that is known to be a poor activating ligand of human TLR9. The modified and unmodified 2006-T4-PDE oligonucleotides were administered in vitro to HEKBlue-hTLR9 cells to determine the immunostimulatory impact of the 3′-cholesteryl modification.

TABLE 3
ODN sequences

| | SEQ ID | |
|---|---|---|
| ODN | NO: | Sequence |
| 3Chol-2006- | SEQ ID | TCGTCGTTTTGTCG |
| T4-PDE | NO: 3 | TTTTGTCGTTTTTT |
| | | X |
| 2006-T4-PDE | SEQ ID | TCGTCGTTTTGTCG |
| | NO: 3 | TTTTGTCGTTTTTT |

X = 3′-Cholesteryl

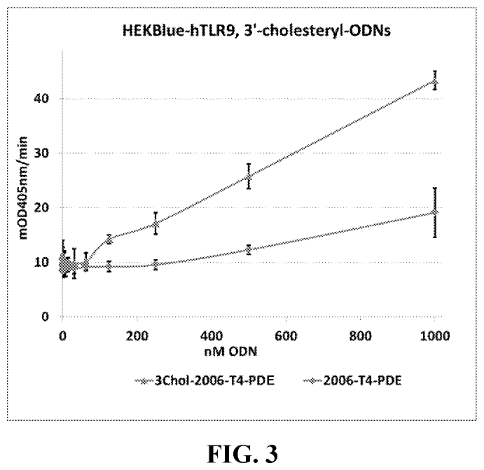

The results suggest that the human TLR9-stimulatory activity of 2006-T4-PDE improves considerably upon 3′-cholesteryl modification (Table 2, FIG. 3).

### 3′-Cholesteryl Modification of PDE-ODNs (III)

3′-cholesteryl modification (see FIG. 1 for the chemical structure of the cholesteryl-linker moiety) was applied to a PDE-ODN that has a fair activity on human TLR9, 2006-3dT4G5T4 (Table 4). The modified and unmodified forms were tested in vitro in HEKBlue-hTLR9 cells.

TABLE 4
ODN sequences

| | SEQ ID | |
|---|---|---|
| ODN | NO: | Sequence |
| 2006- | SEQ ID | TCGTCGTTTTGTCGTT |
| 3dT4G5T4 | NO: 2 | TTGTCGTTTTTTGGGG |
| | | GTTTT |
| 2006- | SEQ ID | TCGTCGTTTTGTCGTT |
| 3dT4G5T43C | NO: 2 | TTGTCGTTTTTTGGGG |
| | | GTTTTX |

X = 3′-Cholesteryl

TABLE 5
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | 81.1 | 55 |
| 2006-3dT4G5T43C | 24.3 | 58 |

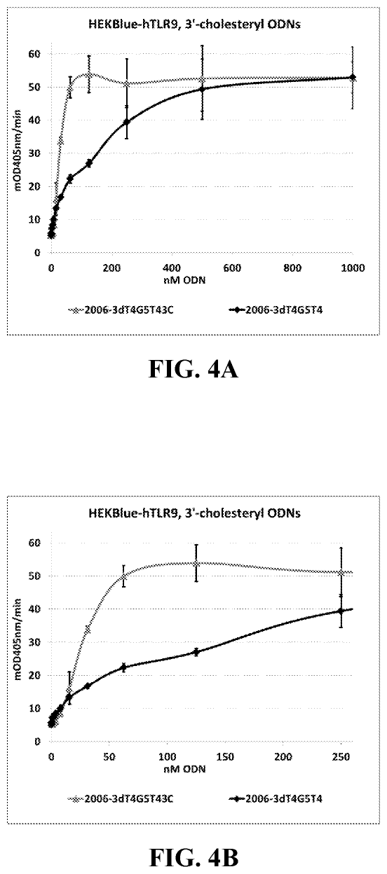

The results suggest that the TLR9-stimulatory activity of 2006-3dT4G5T4 improves considerably upon 3′-cholesteryl modification with respect to the EC₅₀, which is more than 3-fold lower for 2006-3dT4G5T43C (Table 5, FIGS. 4A, 4B).

### 3′-Cholesteryl Modification of PDE-ODNs (IV)

3′-cholesteryl modification (see FIG. 1 for the chemical structure of the cholesteryl-linker moiety) was applied to a PDE-ODN that has a fair activity on human TLR9, 2006-3dT4G5T4 (Table 8). The modified and unmodified forms were tested in vitro in HEKBlue-hTLR9 cells.

TABLE 8
ODN sequences

| | SEQ ID | |
|---|---|---|
| ODN | NO | Sequence |
| 2006-3dT4G5T4 | SEQ ID | TCGTCGTTTTGTCGTT |
| | NO: 2 | TTGTCGTTTTTTGGGG |
| | | GTTTT |
| 2006-3dT4G5T43C | SEQ ID | TCGTCGTTTTGTCGTT |
| | NO: 2 | TTGTCGTTTTTTGGGG |
| | | GTTTTX |

X = 3′-Cholesteryl

TABLE 9
Half Maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | 1175 | 175 |
| 2006-3dT4G5T43C | 32.0 | 56 |

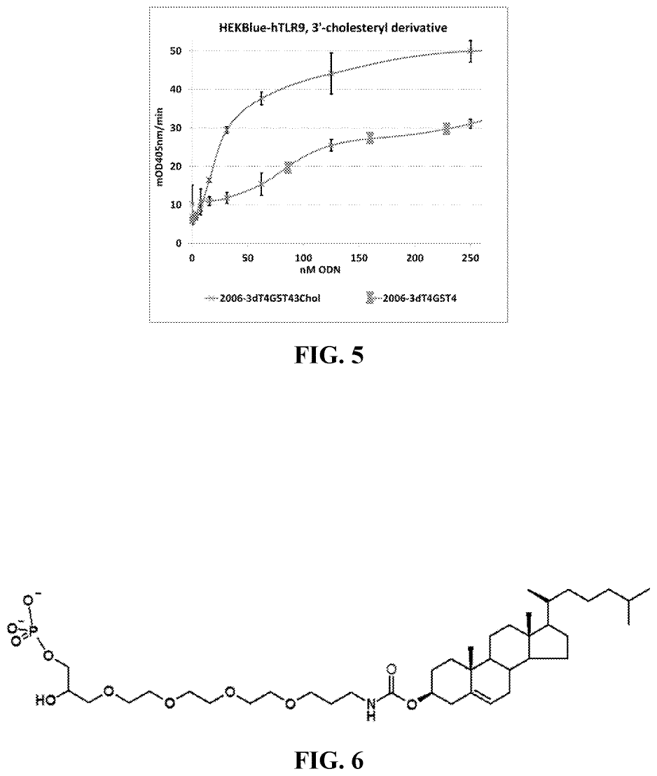

The results suggest that the TLR9-stimulatory activity of 2006-3dT4G5T4 improves considerably upon 3′-cholesteryl modification with respect to the EC₅₀, which is more than 36-fold lower for 2006-3dT4G5T43C (Table 9, FIG. 5).

### 3′-Cholesteryl Modification of PDE-ODNs (V)

3′-cholesteryl modification (see FIG. 6 for the chemical structure of the cholesteryl-linker moiety) applied to a PDE-ODN has a very poor activity on human TLR9, 2006-3dT4G5T4 (Table 10). The modified and unmodified forms were tested in vitro in HEKBlue-hTLR9 cells.

TABLE 10
ODN sequences (lower case: PTO bonds)

| | SEQ ID | |
|---|---|---|
| ODN | NO: | Sequence |
| 2006- | SEQ ID | TCGTCGTTTTGTCGTTTTG |
| 3dT4G5T4 | NO: 2 | TCGTTTTTTGGGGGTTTT |
| 2006-3dT4G5T4- | SEQ ID | TCGTCGTTTTGTCGTTTTG |
| 3Chol | NO: 2 | TCGTTTTTTGGGGGTTTTX |

X = 3′-Cholesteryl

TABLE 11
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | Poorly active | — |
| 2006-3dT4G5T4- | 68.0 | 21 |
| 3Chol | | |

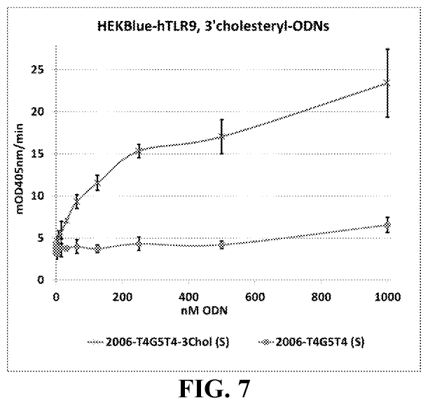

The results suggest that the TLR9-stimulatory activity of 2006-3dT4G5T4 improves massively upon 3′-cholesteryl modification, from virtually nil to an EC₅₀ of 68 nM (Table 11, FIG. 7).

### 3′-Cholesteryl Modification of PDE-ODNs (VI)

3′-cholesteryl modification (see FIG. 1 for the chemical structure of the cholesteryl-linker moiety) was applied to PDE-ODNs that have very poor activity or no activity on human TLR9, 2007-PDE-T4 or 2007-PDE-T4G5T4 (Table 12). The modified and unmodified forms were tested in vitro in HEKBlue-hTLR9 cells.

TABLE 12
ODN sequences

| | SEQ ID | |
|---|---|---|
| ODN | NO | Sequence |
| 2007-PDE-T4 | SEQ ID | TCGTCGTTGTCGTT |
| | NO: 4 | TTGTCGTTTTTT |
| 2007-PDE-T4- | SEQ ID | TCGTCGTTGTCGTT |
| 3Ch | NO: 4 | TTGTCGTTTTTTX |
| 2007-T4G5T4 | SEQ ID | TCGTCGTTGTCGTT |
| | NO: 5 | TTGTCGTTTTTTGG |
| | | GGGTTTT |
| 2007-T4G5T4- | SEQ ID | TCGTCGTTGTCGTT |
| 3Ch | NO: 5 | TTGTCGTTTTTTGG |
| | | GGGTTTTX |

X = 3′-Cholesteryl

TABLE 13
Calculations of effective concentration 50% (EC₅₀₋) and maximum
signal velocity (Vₘₐₓ):

| | EC₅₀ nanomolar | Vmax milliOD 405 nm/min |
|---|---|---|
| ODN | (nM) | (mOD405/min) |
| 2007-PDE-T4 | Inactive | — |
| 2007-PDE-T4-3Ch | Active | — |
| 2007-T4G5T4 | Weakly active | — |
| 2007-T4G5T4-3Ch | 24.9 | 30 |

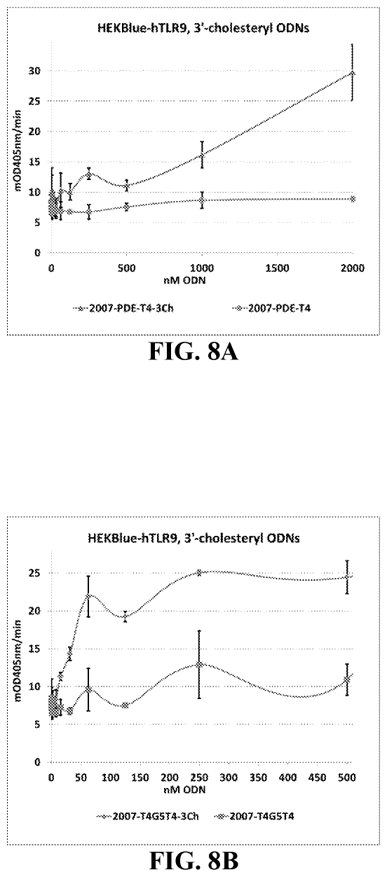

The results suggest that the TLR9-stimulatory activity of both 2007-PDE-T4 and 2007-PDE-T4G5T4 improve massively upon 3′-cholesteryl modification (FIGS. 8A and 8B), in the case 2007-T4G5T4-3Ch to an EC₅₀ of 24.9 nM (Table 13).

### Example 2: Human TLR9, Natural Expression in Ramos-Blue Cells

### 3′-Cholesteryl Modification (I)

3′-cholesteryl modification (see FIG. 1 for the chemical structure of the cholesteryl-linker moiety) was applied to a PDE-ODN that has a fair activity on human TLR9, 2006-3dT4G5T4 (Table 14). The modified and unmodified forms were tested in vitro in Ramos-Blue cells. The Ramos-Blue B lymphocyte cell line (Invivogen, San Diego, CA) stably expresses an NF-κB/AP-1-inducible reporter gene, which allows for the detection of TLR9 signaling.

TABLE 14
ODN sequences (lower case: PTO bonds)

| | SEQ ID | |
|---|---|---|
| ODN | NO | Sequence |
| 2006- | SEQ ID | TCGTCGTTTTGTCGT |
| 3dT4G5T4 | NO: 2 | TTTGTCGTTTTTTGG |
| | | GGGTTTT |
| 2006- | SEQ ID | TCGTCGTTTTGTCGT |
| 3dT4G5T43C | NO: 2 | TTTGTCGTTTTTTGG |
| | | GGGTTTTX |
| 2006-PTO | SEQ ID | tcgtcgttttgtcgt |
| | NO: 1 | tttgtcgtt |

X = 3′-Cholesteryl

TABLE 15
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | 862 | 5.8 |
| 2006-3dT4G5T43C | 107 | 3.7 |
| 2006-PTO | 462 | 4.4 |

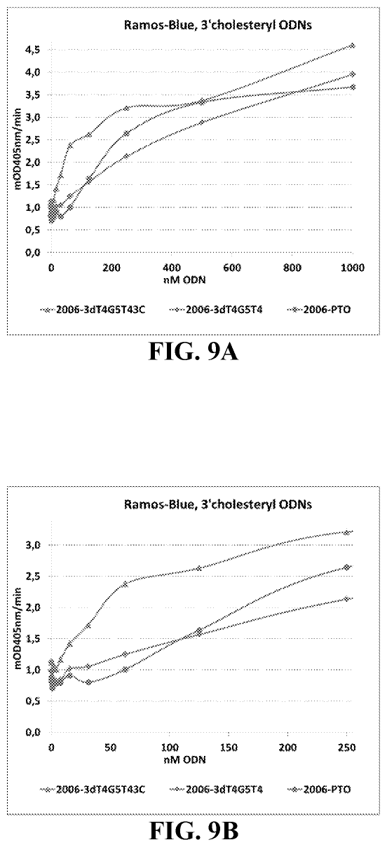

The results suggest that the TLR9-stimulatory activity of 2006-3dT4G5T4 improves considerably upon 3′-cholesteryl modification with respect to the EC₅₀, which is more than 8-fold lower for 2006-3dT4G5T43C (Table 15, FIGS. 9A, 9B). Also, the modified ODN 2006-3dT4G5T43C surpasses the activity of the “industry standard” ODN 2006-PTO.

### 3′-Cholesteryl Modification (II)

3′-cholesteryl modification (see FIG. 1 for the chemical structure of the cholestery-linker group) was applied to a PDE-ODN that has a fair activity on human TLR9, 2006-3dT4G5T4 (Table 16). The modified and unmodified forms were tested in vitro in Ramos-Blue cells.

TABLE 16
ODN sequences

| | SEQ ID | |
|---|---|---|
| ODN | NO | Sequence |
| 2006- | SEQ ID | TCGTCGTTTTGTCGTTTT |
| 3dT4G5T4 | NO: 2 | GTCGTTTTTTGGGGGTTT |
| | | T |
| 2006- | SEQ ID | TCGTCGTTTTGTCGTTTT |
| 3dT4G5T43C | NO: 2 | GTCGTTTTTTGGGGGTTT |
| | | TX |

X = 3′-Cholesteryl

TABLE 17
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | 44.3 | 6.7 |
| 2006-3dT4G5T43C | 331 | 10.7 |

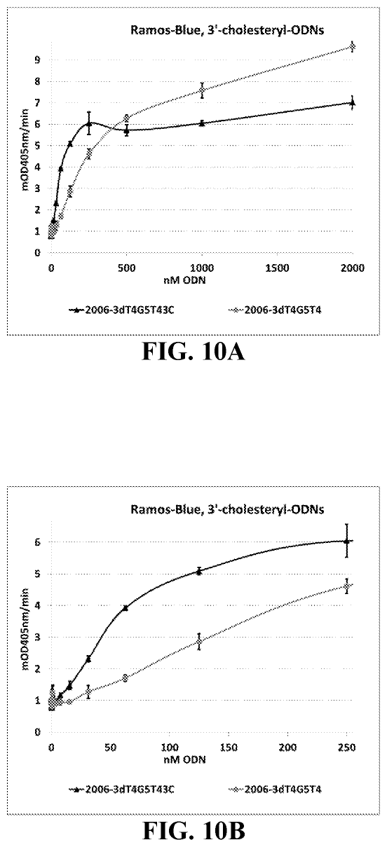

The results suggest that the TLR9-stimulatory activity of 2006-3dT4G5T4 improves considerably upon 3′-cholesteryl modification with respect to the EC₅₀, which is more than 7-fold lower for 2006-3dT4G5T43C (Table 17, FIGS. 10A, 10B).

### 3′-Cholesteryl Modification of PDE-ODNs (III)

3′-cholesteryl modification (see FIG. 1 for the chemical structure of the cholesteryl-linker moiety) was applied to a PDE-ODN that is only poorly active on human TLR9, 2006-T4-PDE (Table 18). The modified and unmodified forms were tested in vitro in Ramos-Blue cells.

TABLE 18
ODN sequences

| | SEQ ID | |
|---|---|---|
| ODN | NO | Sequence |
| 3Chol-2006- | SEQ ID | TCGTCGTTTTGT |
| T4-PDE | NO: 3 | CGTTTTGTCGTT |
| | | TTTTX |
| 2006-T4-PDE | SEQ ID | TCGTCGTTTTGT |
| | NO: 3 | CGTTTTGTCGTT |
| | | TTTT |

X = 3′-Cholesteryl

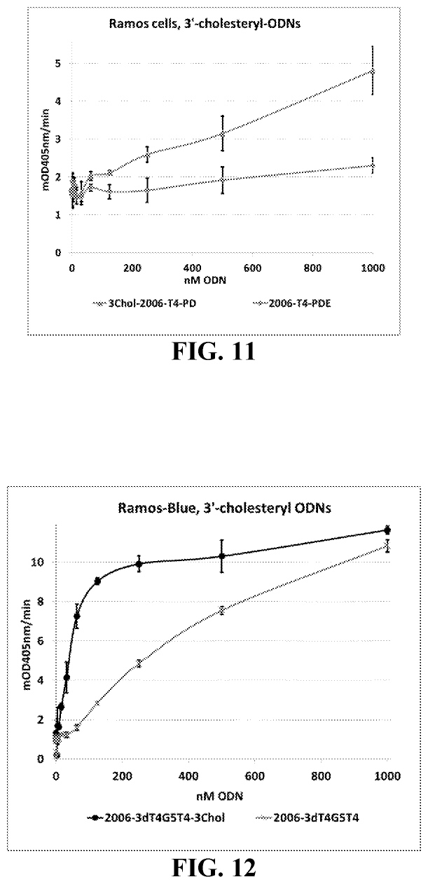

The results suggest that the human TLR9-stimulatory activity of 2006-T4-PDE improves considerably upon 3′-cholesteryl modification (Table 18, FIG. 11).

### 3′-Cholesteryl Modification of PDE-ODNs (IV)

3′-cholesteryl modification (see FIG. 6 for chemical structure of the cholesteryl-linker moiety) was applied to a PDE-ODN that has a very poor activity on human TLR9, 2006-3dT4G5T4 (Table 19). The modified and unmodified forms were tested in vitro in Ramos-Blue cells.

TABLE 19
ODN sequences (lower case: PTO bonds)

| | SEQ ID | |
|---|---|---|
| ODN | NO | Sequence |
| 2006- | SEQ ID | TCGTCGTTTTGTCGTTTT |
| 3dT4G5T4 | NO: 2 | GTCGTTTTTTGGGGGTTTT |
| 2006-3dT4G5T4- | SEQ ID | TCGTCGTTTTGTCGTTTT |
| 3Chol | NO: 2 | GTCGTTTTTTGGGGGTTTTX |

X = 3′-Cholesteryl

TABLE 20
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-3dT4G5T4 | 610 | 17.2 |
| 2006-3dT4G5T4- | 46.3 | 11.9 |
| 3Chol | | |

The results suggest that the human TLR9-stimulatory activity of 2006-3dT4G5T4 on Ramos-Blue cells improves considerably upon 3′-cholesteryl modification (Table 20, FIG. 12) by a factor of 13 with respect to EC₅₀.

### 3′-Cholesteryl Modification of PDE-ODNs (V)

3′-cholesteryl modification (see FIG. 1 for the chemical structure of the cholesteryl-linker moiety) was applied to PDE-ODNs that have very poor activity or no activity on human TLR9, 2007-PDE-T4 or 2007-PDE-T4G5T4 (Table 21). The modified and unmodified forms were tested in vitro in Ramos-Blue cells.

TABLE 21

| | SEQ ID | |
|---|---|---|
| ODN | NO | Sequence |
| 2007-PDE-T4- | SEQ ID | TCGTCGTTGT |
| | NO: 4 | CGTTTTGTCG |
| | | TTTTTT |
| 2007-PDE-T4- | SEQ ID | TCGTCGTTGT |
| 3Ch | NO: 4 | CGTTTTGTCG |
| | | TTTTTTX |
| 2007-T4G5T4 | SEQ ID | TCGTCGTTGT |
| | NO: 5 | CGTTTTGTCG |
| | | TTTTTTGGGG |
| | | GTTTT |
| 2007-T4G5T4- | SEQ ID | TCGTCGTTGT |
| 3Ch | NO: 5 | CGTTTTGTCG |
| | | TTTTTTGGGG |
| | | GTTTTX |
| TCG8-T4 | SEQ ID | TCGTCGTCGT |
| | NO: 6 | CGTCGTCGTC |
| | | GTCGTTTT |
| TCG8-T4-3Ch | SEQ ID | TCGTCGTCGT |
| | NO: 6 | CGTCGTCGTC |
| | | GTCGTTTTX |

X = 3′-Cholesteryl

TABLE 22
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2007-PDE-T4 | — | — |
| 2007-PDE-T4-3Ch | 1219 | 17.2 |
| 2007-T4G5T4 | 401 | 22.5 |
| 2007-T4G5T4-3Ch | 28.7 | 14.1 |
| TCG8-T4 | — | — |
| TCG8-T4-3Ch | 362 | 9.7 |

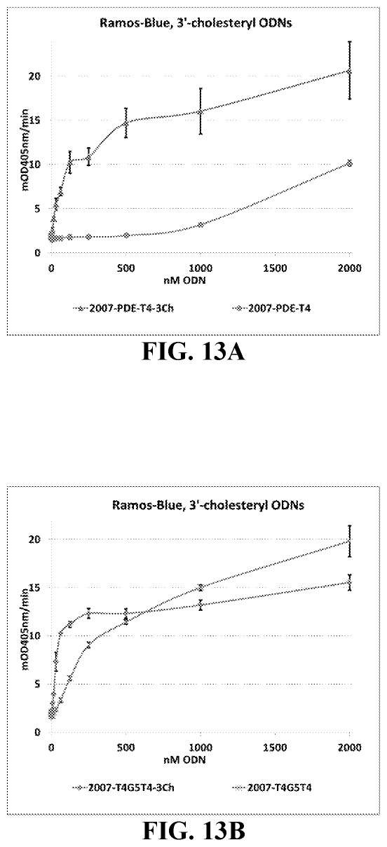

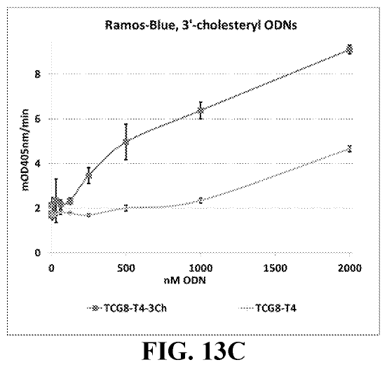

The results suggest that the human TLR9-stimulatory activity of 2006-3dT4G5T4 in Ramos-Blue cells improves considerably upon 3′-cholesteryl modification of all ODNs considered in this experiment (2007-PDE-T4, 2007-T4G5T4, TCG8-T4, Table 22, FIGS. 13A, 13B and 13C). In the case of 2007-T4G5T4-Ch, an improvement of activity by a factor of almost 14 with respect to EC₅₀ was noted compared to its non-modified congener.

### Example 3: Mouse TLR9, Recombinant Overexpression in HEKBlue

### 3′-Cholesteryl Modification of PDE-ODNs

3′-cholesteryl modification (see FIG. 1 for the chemical structure of the cholesteryl-linker moiety) was applied to 2007-PDE-T4, 2007-PDE-T4G5T4, and TCG8-T4 that have a very poor activity or no activity on human TLR9 (Table 23). The modified and unmodified forms were tested in vitro in HEKBlue-mTLR9 cells (Invivogen).

TABLE 23
ODN sequences

| | SEQ ID | |
|---|---|---|
| ODN | NO | Sequence |
| 2007-PDE-T4 | SEQ ID | TCGTCGTTGT |
| | NO: 4 | CGTTTTGTCG |
| | | TTTTTT |
| 2007-PDE-T4- | SEQ ID | TCGTCGTTGT |
| 3Ch | NO: 4 | CGTTTTGTCG |
| | | TTTTTTX |
| 2007-T4G5T4 | SEQ ID | TCGTCGTTGT |
| | NO: 5 | CGTTTTGTCG |
| | | TTTTTTGGGG |
| | | GTTTT |
| 2007-T4G5T4- | SEQ ID | TCGTCGTTGT |
| 3Ch | NO: 5 | CGTTTTGTCG |
| | | TTTTTTGGGG |
| | | GTTTTX |
| TCG8-T4 | SEQ ID | TCGTCGTCGT |
| | NO: 6 | CGTCGTCGTC |
| | | GTCGTTTT |
| TCG8-T4-3Ch | SEQ ID | TCGTCGTCGT |
| | NO: 6 | CGTCGTCGTC |
| | | GTCGTTTTX |
| TCG8-T4G5T4 | SEQ ID | TCGTCGTCGT |
| | NO: 8 | CGTCGTCGTC |
| | | GTCGTTTTGG |
| | | GGGTTTT |
| TCG8-T4G5T4- | SEQ ID | TCGTCGTCGT |
| 3Chol | NO: 8 | CGTCGTCGTC |
| | | GTCGTTTTGG |
| | | GGGTTTTX |

X = 3′-Cholesteryl

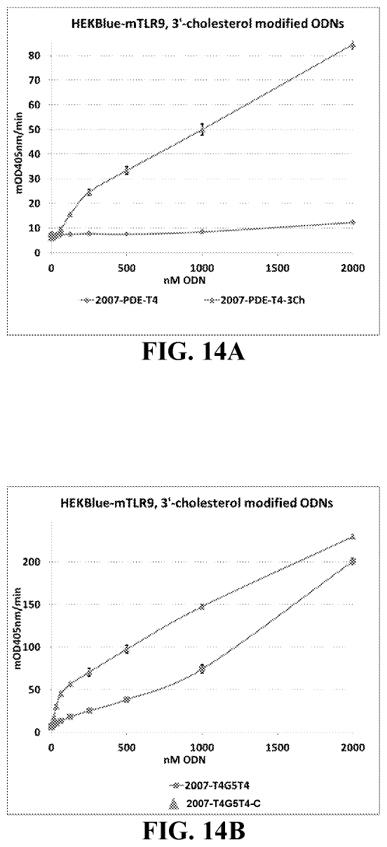

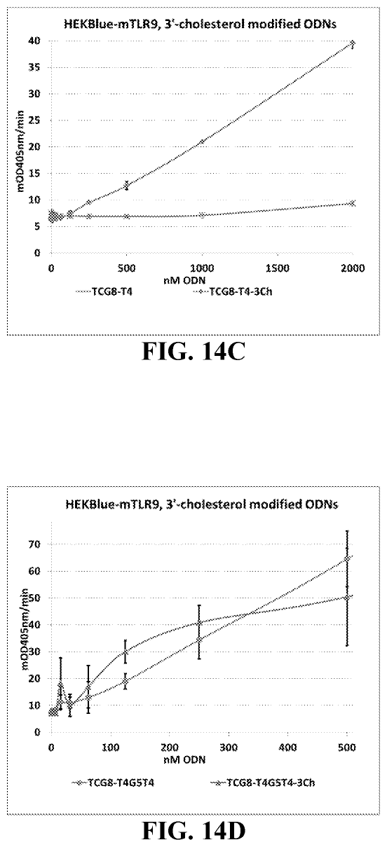

The results suggest that the mouse TLR9-stimulatory activity of all ODNs considered in this experiment (2007-PDE-T4, 2007-T4G5T4, TCG8-T4, Table 23) in HEKBlue-mTLR9 improves considerably upon 3′-cholesteryl modification in three cases (FIGS. 14A, 14B, and 14C), and marginally at low concentrations in a fourth example (FIG. 14D).

### Example 4: Systematic Study on Unmodified, 3′-Cholesteryl Modified and 5′-Cholesteryl Modified ODNs on HEKBlue-hTLR9 and Ramos-Blue Cells: Structure-Activity Relationship (SAR)

### HEKBlue-hTLR9

TABLE 24
ODN sequences (lower case
indicates PTO bonds)

| | SEQ ID | |
|---|---|---|
| ODN | NO | Sequence |
| 2006-PTO | SEQ ID | tcgtcgtttt |
| | NO: 1 | gtcgttttgt |
| | | cgtt |
| 2006-PDE-T4 | SEQ ID | TCGTCGTTTT |
| | NO: 3 | GTCGTTTTGT |
| | | CGTTTTTT |
| 2006-PDE-T4- | SEQ ID | TCGTCGTTTT |
| 3Chol | NO: 3 | GTCGTTTTGT |
| | | CGTTTTTTX |
| 2006-PDE-T4- | SEQ ID | YTCGTCGTTT |
| 5Chol | NO: 3 | TGTCGTTTTG |
| | | TCGTTTTTT |
| 2006-G5 | SEQ ID | TCGTCGTTTT |
| | NO: 7 | GTCGTTTTGT |
| | | CGTTGGGGG |
| 2006-G5- | SEQ ID | TCGTCGTTTT |
| 3Chol | NO: 7 | GTCGTTTTGT |
| | | CGTTGGGGGX |
| 2006-G5- | SEQ ID | YTCGTCGTTT |
| 5Chol | NO: 7 | TGTCGTTTTG |
| | | TCGTTGGGGG |
| 2006-T4G5T4 | SEQ ID | TCGTCGTTTT |
| | NO: 2 | GTCGTTTTGT |
| | | CGTTTTTTGG |
| | | GGGTTTT |
| 2006-T4G5T4- | SEQ ID | TCGTCGTTTT |
| 3Chol | NO: 2 | GTCGTTTTGT |
| | | CGTTTTTTGG |
| | | GGGTTTTX |
| 2006-T4G5T4- | SEQ ID | YTCGTCGTTT |
| 5Chol | NO: 2 | TGTCGTTTTG |
| | | TCGTTTTTTG |
| | | GGGGTTTT |
| TCG8-T4G5T4 | SEQ ID | TCGTCGTCGT |
| | NO: 8 | CGTCGTCGTC |
| | | GTCGTTTTGG |
| | | GGGTTTT |
| TCG8-T4G5T4- | SEQ ID | TCGTCGTCGT |
| 3Chol | NO: 8 | CGTCGTCGTC |
| | | GTCGTTTTGG |
| | | GGGTTTTX |
| TCG8-T4G5T4- | SEQ ID | YTCGTCGTCG |
| 5Chol | NO: 8 | TCGTCGTCGT |
| | | CGTCGTTTTG |
| | | GGGGTTTT |

X = 3′-Cholesteryl
Y = 5′-Cholesteryl

TABLE 25
Half maximum effective concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-PTO | 586 | 55 |
| 2006-PDE-T4 | Very weak | — |
| 2006-PDE-T4-3Chol | Active (linear) | — |
| 2006-PDE-T4-5Chol | Inactive | — |
| 2006-G5 | 362 | 30 |
| 2006-G5-3Chol | 144 | 30 |
| 2006-G5-5Chol | Very weak | — |
| 2006-T4G5T4 | 1133 | 69 |
| 2006-T4G5T4-3Chol | 23.3 | 26 |
| 2006-T4G5T4-5Chol | Very weak | — |
| TCG8-T4G5T4 | Weak | — |
| TCG8-T4G5T4-3Chol | Weak | — |
| TCG8-T4G5T4-5Chol | Weak | — |

In this experiment, the zero ODN values were subtracted from every data point for the EC50 and Vmax calculation, due to relatively high background readings.

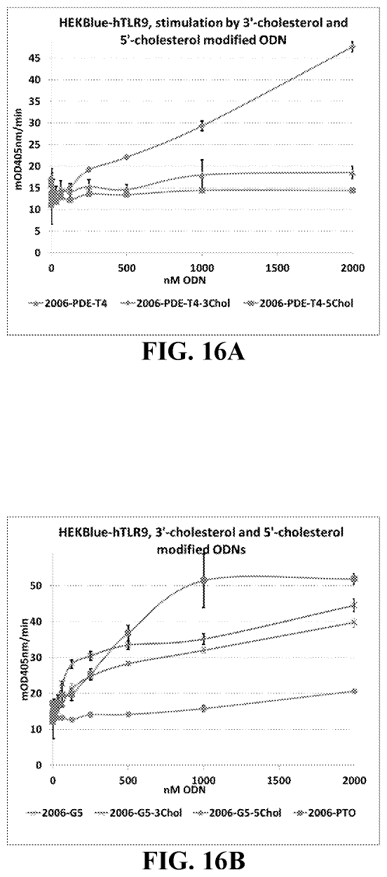

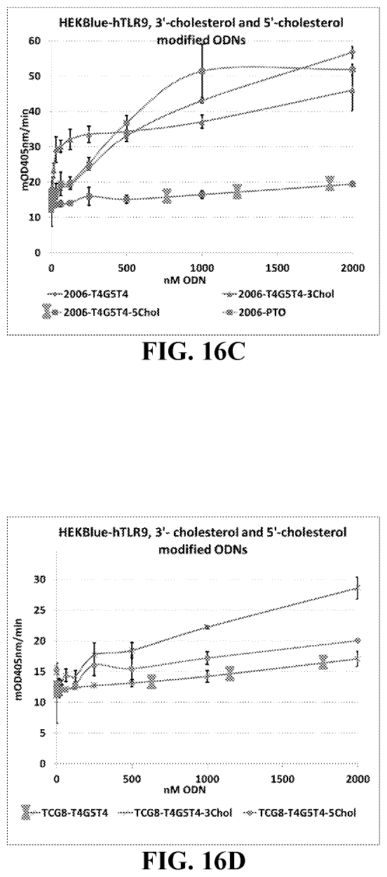

For every ODN investigated in this experiment, the 3′-cholesteryl modification was most beneficial to activity on human TLR9 expressed in HEKblue cells (Table 24, FIGS. 16A, 16B, 16C, 16D). In those cases where EC₅₀/Vₘₐₓ calculations were possible (2006-G5, 2006-T4G5T4, Table 25), it was found that the EC₅₀ of the unmodified ODN was lower (factors of 2.5 and 48, respectively), while 5′-cholesteryl modification led to activity loss. The EC₅₀s of 2006-G5-3Chol and 2006-T4G5T4-3Chol were lower than those of the “industry standard” 2006-PTO, making them candidates for immunomodulatory intervention.

### Ramos-Blue

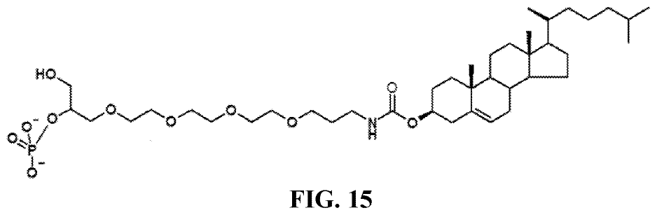

3′-cholesteryl or 5′-cholesteryl modifications (see FIGS. 6 and 15, respectively for the chemical structures of the cholesteryl-linker moietys) were applied to 4 different ODNs (Table 26). The modified and unmodified forms were tested in vitro in Ramos-Blue cells.

TABLE 26
ODN sequences (lower case:
PTO bonds)

| | SEQ ID | |
|---|---|---|
| ODN | NO | Sequence |
| 2006-PTO | SEQ ID | tcgtcgtttt |
| | NO: 1 | gtcgttttgt |
| | | cgtt |
| 2006-PDE-T4 | SEQ ID | TCGTCGTTTT |
| | NO: 3 | GTCGTTTTGT |
| | | CGTTTTTT |
| 2006-PDE-T4- | SEQ ID | TCGTCGTTTT |
| 3Chol | NO: 3 | GTCGTTTTGT |
| | | CGTTTTTTX |
| 2006-PDE-T4- | SEQ ID | YTCGTCGTTT |
| 5Chol | NO: 3 | TGTCGTTTTG |
| | | TCGTTTTTT |
| 2006-G5 | SEQ ID | TCGTCGTTTT |
| | NO: 7 | GTCGTTTTGT |
| | | CGTTGGGGG |
| 2006-G5- | SEQ ID | TCGTCGTTTT |
| 3Chol | NO: 7 | GTCGTTTTGT |
| | | CGTTGGGGGX |
| 2006-G5- | SEQ ID | YTCGTCGTTT |
| 5Chol | NO: 7 | TGTCGTTTTG |
| | | TCGTTGGGGG |
| 2006-T4G5T4 | SEQ ID | TCGTCGTTTT |
| | NO: 2 | GTCGTTTTGT |
| | | CGTTTTTTGG |
| | | GGGTTTT |
| 2006-T4G5T4- | SEQ ID | TCGTCGTTTT |
| 3Chol | NO: 2 | GTCGTTTTGT |
| | | CGTTTTTTGG |
| | | GGGTTTTX |
| 2006-T4G5T4- | SEQ ID | YTCGTCGTTT |
| 5Chol | NO: 2 | TGTCGTTTTG |
| | | TCGTTTTTTG |
| | | GGGGTTTT |
| TCG8-T4G5T4 | SEQ ID | TCGTCGTCGT |
| | NO: 8 | CGTCGTCGTC |
| | | GTCGTTTTGG |
| | | GGGTTTT |
| TCG8-T4G5T4- | SEQ ID | TCGTCGTCGT |
| 3Chol | NO: 8 | CGTCGTCGTC |
| | | GTCGTTTTGG |
| | | GGGTTTTX |
| TCG8-T4G5T4- | SEQ ID | YTCGTCGTCG |
| 5Chol | NO: 8 | TCGTCGTCGT |
| | | CGTCGTTTTG |
| | | GGGGTTTT |

X = 3′-Cholesteryl
Y = 5′-Cholesteryl

TABLE 27
Half maximum concentration (EC₅₀) and maximum
signal velocity (Vₘₐₓ)

| | EC₅₀ nanomolar | Vmax milliOD 405 nm/min |
|---|---|---|
| ODN | (nM) | (mOD405/min) |
| 2006-PTO | 287 | 20 |
| 2006-PDE-T4 | Weak | — |
| 2006-PDE-T4-3Chol | Active (linear) | — |
| 2006-PDE-T4-5Chol | Active (linear) | — |
| 2006-G5 | 771 | 22 |
| 2006-G5-3Chol | 247 | 14 |
| 2006-G5-5Chol | Weak | — |
| 2006-T4G5T4 | 439 | 22 |
| 2006-T4G5T4-3Chol | 42.8 | 14 |
| 2006-T4G5T4-5Chol | Weak | — |
| TCG8-T4G5T4 | Weak | — |
| TCG8-T4G5T4-3Chol | 339 | 9 |
| TCG8-T4G5T4-5Chol | 1438 | 14 |

In this experiment, the zero ODN values were subtracted from every data point for the EC50 and Vmax calculation, for consistency with Table 25).

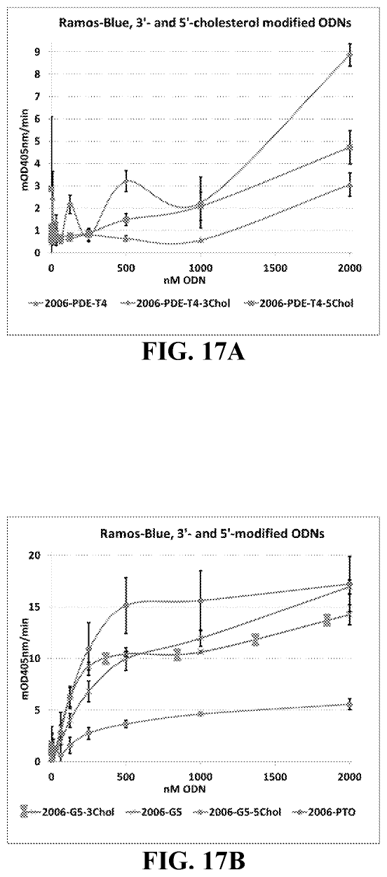

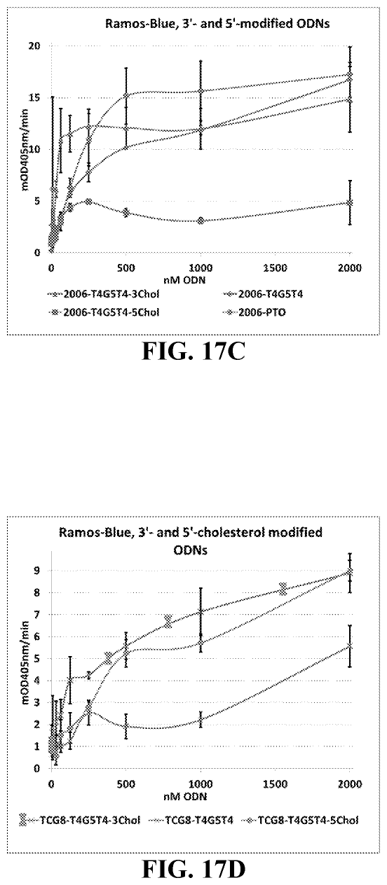

For every ODN investigated in this experiment, the 3′-cholesteryl modification was most beneficial to activity on human TLR9 endogenously present in Ramos-Blue cells (Table 26, FIGS. 17A-17D). In those cases where EC₅₀/Vₘₐₓ calculations were possible (2006-G5, 2006-T4G5T4, Table 25), it was found that the EC₅₀ of the unmodified ODN was lower (factors of 3 and 10, respectively), while 5′-cholesteryl modification led to activity loss, except for TCG8-T4G5T4, where both derivatizations led to improved activity, but more so for 3′-cholesteryl than for 5′-cholesteryl (FIG. 17D, Table 26). The EC₅₀s of 2006-G5-3Chol and 2006-T4G5T4-3Chol were lower than those of the “industry standard” 2006-PTO, making them candidates for immunomodulatory intervention.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed:

1. An immunostimulatory oligonucleotide comprising at least one CpG motif and a 3′ cholesteryl moiety.

2. The immunostimulatory oligonucleotide of claim 1 comprising phosphodiester linkages or phosphorothioate linkages between nucleotides of the immunostimulatory oligonucleotide.

3. The immunostimulatory oligonucleotide of claim 1, wherein the cholesteryl moiety is covalently attached to the 3′-terminal nucleotide of the immunostimulatory oligonucleotide via a linker.

4. The immunostimulatory oligonucleotide of claim 1, further comprising a 3′ terminal sequence comprising a plurality of thymine nucleotides.

5. The immunostimulatory oligonucleotide of claim 4, wherein the plurality of thymine nucleotides comprises consecutive thymine nucleotides.

6. The immunostimulatory oligonucleotide of claim 4, wherein the plurality of thymine nucleotides comprises between 4 and 6 consecutive thymine nucleotides.

7. The immunostimulatory oligonucleotide of claim 1, wherein the 3′ terminal sequence is TTTT.

8. The immunostimulatory oligonucleotide of claim 1, wherein the 3′ terminal sequence of the immunostimulatory oligonucleotide comprises a plurality of guanine nucleotides.

9. The immunostimulatory oligonucleotide of claim 8, wherein the plurality of guanine nucleotides comprises consecutive guanine nucleotides.

10. The immunostimulatory oligonucleotide of claim 8, wherein the 3′ terminal sequence is GGGGG.

11. The immunostimulatory oligonucleotide of claim 1, wherein the immunostimulatory oligonucleotide comprises (TCG)ₙ, where n is between 3 and 10.

12. The immunostimulatory oligonucleotide of claim 4, wherein the linker comprises a carbon chain.

13. The immunostimulatory oligonucleotide of claim 12, wherein the carbon chain comprises between 3 and 12 carbon atoms.

14. The immunostimulatory oligonucleotide of claim 3, wherein the linker comprises a hexanediol.

15. The immunostimulatory oligonucleotide of claim 1, wherein the oligonucleotide further comprises a cholesteryl-linker moiety having the following structure:

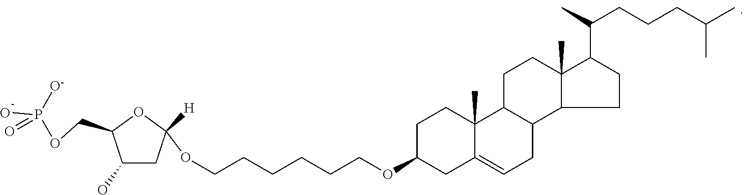

16. The immunostimulatory oligonucleotide of claim 3, wherein the linker comprises a repeated chemical unit, optionally repeated between 2 and 12 times.

17. The immunostimulatory oligonucleotide of claim 16, wherein the repeated chemical unit is an ethylene glycol or hexaethylene glycol.

18. The immunostimulatory oligonucleotide of claim 1, wherein the cholesteryl moiety is covalently bound to the linker to form a cholesteryl-linker moiety having the following structure:

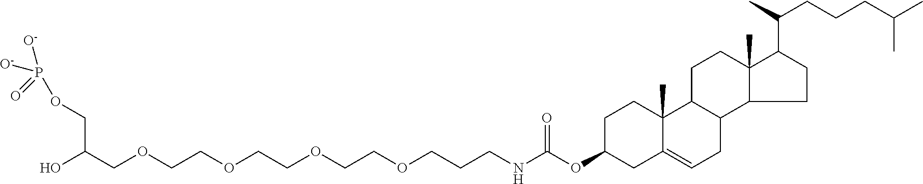

19. An immunostimulatory composition comprising the immunostimulatory oligonucleotide claim 1.

20. A method of eliciting a TLR9-mediated immune response in a subject comprising administering to the subject the immunostimulatory oligonucleotide of claim 1.
